(19) United States
(12) Patent Application Publication
(10) Pub. No.: US 2007/0275926 A1
(43) Pub. Date: Nov. 29, 2007

(54) TREATMENT OF DISORDERS OF THE PERIPHERAL NERVOUS SYSTEM

(75) Inventors: AHMET HOKE, TOWSON, MD (US); RUIFA MI, BALTIMORE, MD (US)

Correspondence Address: BANNER & WITCOFF, LTD., 1100 13th STREET, N.W., SUITE 1200, WASHINGTON, DC, 20005-4051, US

(73) Assignee: The Johns Hopkins University, Baltimore, MD (US)

(21) Appl. No.: 11/752,044

(22) Filed: May 22, 2007

Related U.S. Application Data
(62) Division of application No. 11/262,969, filed on Nov. 1, 2005.
(60) Provisional application No. 60/624,633, filed on Nov. 3, 2004.

(51) Int. Cl. A61K 48/00; A61P 25/02

(52) U.S. Cl. 514/44; 424/93.2

(57) ABSTRACT

Pleiotrophin is used to treat various conditions, disorders, and diseases which involve damage to the peripheral nervous system. Such conditions include traumatic nerve damage, Charcot-Marie-Tooth disease, amyotrophic lateral sclerosis, spinobulbar muscular atrophy, spinal muscular atrophy, diabetic neuropathy, and uremic neuropathy. Pleiotrophin can be provided as a protein or as a gene therapy to a patient in need thereof.

This application claims the benefit of provisional application Ser. No. 60/624,633, filed Nov. 3, 2004, the disclosure of which is expressly incorporated herein.

### TECHNICAL FIELD OF THE INVENTION

This invention is related to the area of disorders of the nervous system. In particular, it relates to disorders of the peripheral nervous system.

### BACKGROUND OF THE INVENTION

Chronic neurodegenerative illnesses that affect the peripheral nervous system (PNS), such as amyotrophic lateral sclerosis (ALS), Charcoat-Marie-Tooth (CMT) diseases or neuropathies such as diabetic neuropathy do not have any effective treatment. Various growth factors that may act as neurotrophic factors are being developed for the treatment of neurodegenerative diseases. However, currently, none of the available neurotrophic factors have been shown to be effective in treating peripheral nervous system illnesses.

There is a continuing need in the art for effective methods for treating these illnesses.

### SUMMARY OF THE INVENTION

According to one embodiment of the invention a method is provided for treating a peripheral neuropathy, peripheral neurodegenerative disease, or peripheral nerve trauma. An effective amount of pleiotrophin is administered to a patient with a peripheral neuropathy, peripheral neurodegenerative disease, or peripheral nerve trauma. Axonal regeneration is thereby stimulated or neuronal death is thereby inhibited.

According to another embodiment of the invention a method is provided for treating a peripheral neuropathy, peripheral neurodegenerative disease, or peripheral nerve trauma. An effective amount of a nucleic acid is administered to a patient with a peripheral neuropathy, peripheral neurodegenerative disease, or peripheral nerve trauma. The nucleic acid encodes pleiotrophin. Axonal regeneration is thereby stimulated or neuronal death is thereby inhibited.

These and other embodiments which will be apparent to those of skill in the art upon reading the specification provide the art with reagents and methods for detection, diagnosis, therapy, and drug screening pertaining to neuronal cell death and pathological processes involving or requiring neuronal cell death.

### BRIEF DESCRIPTION OF THE DRAWINGS

FIG. 1: Pleiotrophin mRNA is upregulated after injury in the adult rodent. The figure shows the time course of pleiotrophin mRNA expression in the distal sciatic nerve after transaction of the left sciatic nerve of adult rats. Levels of mRNA were measured using real-time RT-PCR

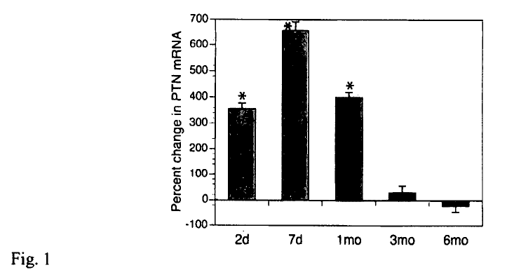

FIG. 2: PTN mRNA is expressed in muscle. The figure shows expression in distal leg muscles harvested at various stages of development and post denervation. PTN mRNA levels were measured using real-time RT-PCR and normalized to GAPDH.

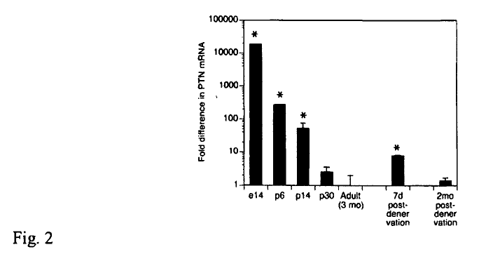

FIG. 3: PTN mRNA is expressed in the skin. Levels of PTN mRNA in plantar footpad skins at different developmental stages and post denervation were measured using real-time RT-PCR, normalized to GAPDH, and expressed as a fold difference from the adult skin levels.

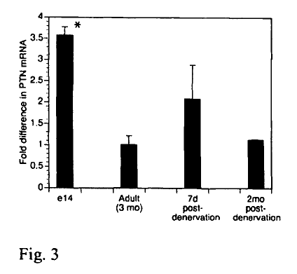

FIG. 4: PTN causes directional outgrowth of motor axons out of spinal cord explants. Directional growth toward PTN-soaked gelfoams of motor axons (stained with SMI-32 monoclonal antibody against neurofilament). There was no directional growth with vehicle-soaked gelfoams.

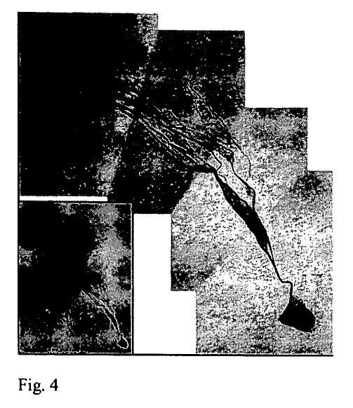

FIG. 5A-5C: PTN induces spinal motor axon regeneration in vitro. HEK-293 cells transfected with PTN (FIG. 5C) or the vector alone (FIG. 5B) were co-cultured with spinal cord explants. The explants were stained with neurofilament antibody (SMI-32) and the number of motor axons crossing the white matter and exiting from the spinal cord explants were counted. The results of the counting are shown in FIG. 5A. (* p<0.005).

FIG. 6: PTN induces axon outgrowth in dorsal root ganglion (DRG) explants. Embryonic day 14 DRG were excised and cultured in media containing rhPTN or in conditioned media from HEK-293 cells transfected with PTN or vector. Explants were stained with anti-neurofilament antibody and the number of axons reaching at least a 100 μm away form the explant were counted (n=4 per group; GF=growth factor; * p<0.05).

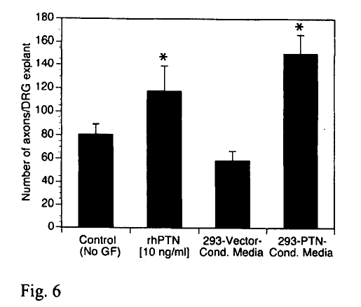

FIG. 7A-7C. PTN induces axonal regeneration in vivo. A sciatic nerve of a rat was transected and repaired with silicone nerve guides, leaving a gap of more than 10 mm between the proximal and distal ends. HEK-293 cells expressing either PTN or control plasmid were transplanted into the silicone tubes. Two months later nerve regeneration was assessed by counting the number of axons that regenerated 15 mm distally into the silicone tube. Results are shown.

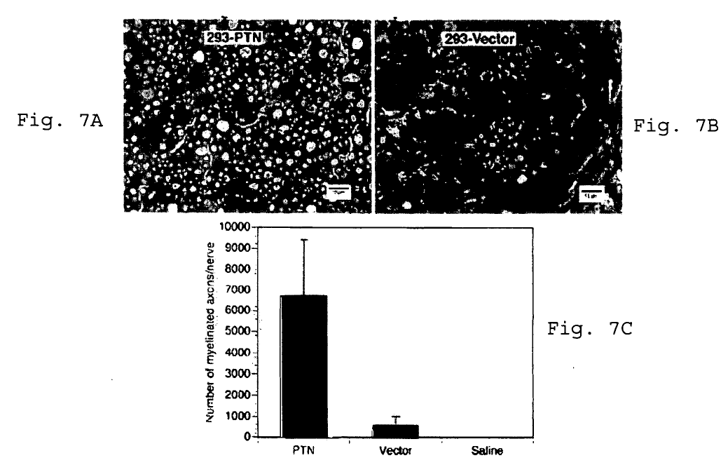

FIG. 8: PTN protects spinal motor neurons against chronic excitotoxic glutamate toxicity. Glutamate transport inhibitor threo-hydroxyaspartate (THA) with and without rhPTN was applied to cultures of postnatal day 8 spinal cord explants for four weeks. Explants were stained with anti-neurofilament antibody and the number of motor neurons was counted. (* p<0.005 between control and THA; ** p<0.005 between THA and THA+PTN). Results are shown.

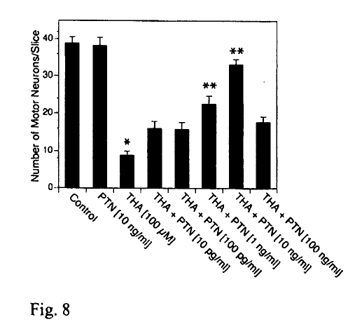

FIG. 9: PTN protects facial motor neurons against growth factor deprivation-induced cell death. Facial axotomies were performed on postnatal day 2 mice and gelfoams soaked with rhPTN were transplanted into the stump of the facial nerve. Brainstems were stained with anti-neurofilament antibody to label the facial motor neurons and the number of remaining facial motor neurons in each facial ganglion was determined. Results are shown.

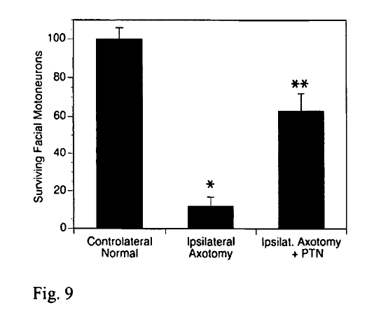

### DETAILED DESCRIPTION OF THE INVENTION

The inventors have found that pleiotrophin (PTN), a growth factor of the cytokine family, is a neurotrophic factor for both the motor and sensory neurons of the PNS. In tissue culture models and in animal models, PTN protects motor and sensory neurons from death and promotes regeneration of their axons. Recombinant human PTN can be developed as a therapeutic tool for a variety of neurodegenerative illnesses affecting the PNS. We have shown that PTN promotes axon regeneration and outgrowth in motor and sensory neurons in vitro and in vivo after nerve transection injury. It also protects motor neurons against chronic excitotoxic injury in vitro and neurotrophic factor deprivation induced neuronal death in vivo.

The therapeutic treatment according to the present invention can be used for both inherited and acquired neurodegenerative diseases in the PNS. These potential therapeutic targets can range from rare diseases such as ALS, and CMT to more common diseases such as diabetic neuropathy.

PTN (UniGene Hs. 371249) is also known as heparin binding growth factor 8, and neurite growth-promoting factor 1). Coding sequences for PTN are available at GenBank as M57399.1 Human nerve growth factor (HBNF-1) mRNA, complete cds; NM_{—}002825.5 Homo sapiens pleiotrophin (heparin binding growth factor 8, neurite growth-promoting factor 1) (PTN), mRNA; CR450338.1 Homo sapiens full open reading frame cDNA clone RZPDo834C092D for gene PTN, pleiotrophin (heparin binding growth factor 8, neurite growth-promoting factor 1); complete cds; without stopcodon; CR624136.1 full-length cDNA clone CS0DB007YG20 of Neuroblastoma Cot 10-normalized of Homo sapiens (human); CR620419.1 full-length cDNA clone CS0DI004YO21 of Placenta Cot 25-normalized of Homo sapiens (human); CR619084.1 full-length cDNA clone CS0DF008YL14 of Fetal brain of Homo sapiens (human); CR614046.1 full-length cDNA clone CS0DF009YH21 of Fetal brain of Homo sapiens (human); CR609152.1 full-length cDNA clone CL0BB026ZD05 of Neuroblastoma of Homo sapiens (human); CR596476.1 full-length cDNA clone CS0DI066YB11 of Placenta Cot 25-normalized of Homo sapiens (human); BT019692.1 Homo sapiens pleiotrophin (heparin binding growth factor 8, neurite growth-promoting factor 1) mRNA, complete cds; BC005916.1 Homo sapiens pleiotrophin (heparin binding growth factor 8, neurite growth-promoting factor 1), mRNA (cDNA clone MGC:14512 IMAGE:4248708), complete cds; X52946.1 Human pleiotrophin (PTN) mRNA; and D90226.1 Homo sapiens osf-1 mRNA, complete cds. Any of these coding sequences can be used to provide PTN to a subject in need thereof. Coding sequences that are at least 95%, 96%, 97%, 98%, or 99% identical can also be used. Any of the encoded sequences of these mRNA molecules can be used as protein therapeutics. Exemplary of such protein sequences are those in the NCBI database as accession numbers CAA37121, AAH05916, NP_{—}002816, and AAB24425. Any of these or other naturally occurring or synthetic variants having at least 95%, 96%, 97%, 98%, or 99% identity to the database sequences can be used. Any database sequence cited herein, refers to the sequence as it existed on the filing date of the subject application. Fusion proteins comprising PTN or other modifications to its structure that retain function can be used as well. Such modifications can include detectable labels and moieties for monitoring, for example.

The proteins or nucleic acids which can be used to supply PTN function to a subject with a peripheral nerve disorder can be formulated according to any known method for such protein and nucleic acid molecules in general. Protective, stabilizing, or slow-release formulations can be used, as are known in the art. Typically the proteins and nucleic acids will be administered as liquids, i.e., in a carrier or vehicle. Gels and semisolids can also be used for particular applications. Some formulations will provide suitable qualities for use as an injectable, e.g., in a pyrogen-free and sterile carrier or vehicle. Nanoparticles, liposomes, polymer carriers, and emulsions, are examples of suitable vehicles.

PTN protein or PTN-encoding nucleic acids can be delivered according to any method known in the art. These include, without limitation, subcutaneous, intramuscular, intrathecal, transdermal, intravenous, oral, sublingual, nasal, rectal, and topical administrations.

Vectors which can be used to deliver a PTN encoding nucleic acid include viral and non-viral vectors. Suitable vectors which can be used include adenovirus, adeno-associated virus, retrovirus, lentivirus, HSV (herpes simplex virus) and plasmids. If the vector is in a viral vector and the vector has been packaged, then the virions can be used to infect cells. If naked DNA is used, then transfection or transformation procedures as are appropriate for the particular host cells can be used. Formulations of naked DNA utilizing polymers, liposomes, or nanospheres can be used for gene delivery. Nucleic acids can be administered in any desired format that provides sufficiently efficient delivery levels, including in virus particles, in liposomes, in nanoparticles, and complexed to polymers.

Pharmaceutical formulations suitable for parenteral administration can be formulated in aqueous solutions, preferably in physiologically compatible buffers such as Hanks' solution, Ringer's solution, or physiologically buffered saline. Aqueous injection suspensions can contain substances that increase the viscosity of the suspension, such as sodium carboxymethyl cellulose, sorbitol, or dextran. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Non-lipid polycationic amino polymers also can be used for delivery. Optionally, the suspension also can contain suitable stabilizers or agents that increase the solubility of the compounds to allow for the preparation of highly concentrated solutions. For topical or nasal administration, penetrants appropriate to the particular barrier to be permeated are used in the formulation. Such penetrants are generally known in the art.

The pharmaceutical compositions for use in the present invention can be manufactured in a manner that is known in the art, e.g., by means of conventional mixing, dissolving, granulating, dragee-making, levigating, emulsifying, encapsulating, entrapping, or lyophilizing processes. The pharmaceutical composition can be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms.

Further details on techniques for formulation and administration are discussed in, for example, Hoover, John E., Remington's Pharmaceutical Sciences, Mack Publishing Co., Easton, Pa., 1975; Liberman, et al., Eds., Pharmaceutical Dosage Forms, Marcel Decker, New York, N.Y., 1980; and Kibbe, et al., Eds., Handbook of Pharmaceutical Excipients (3^{rd} Ed.), American Pharmaceutical Association, Washington, 1999; U.S. Pharamacopeia (Twenty-First Revision—USP XXI) National Formulary (Sixteenth Edition—XVI), United States Pharmacopeial Convention, Inc., Rockville, Md., 1985, and its later editions; and Remington's Pharmaceutical Sciences, 16^{th} Edition, Arthur Osol, Editor and Chairman of the Editorial Board, Mack Publishing Co., Easton, Pa., 1980, and its later editions.

The PTN proteins and nucleic acids of the present invention can be used to treat peripheral neurological disorders and may be administered by any suitable route and in a therapeutically effective dose for the treatment intended. The active compounds and compositions, for example, may be administered orally, sublingually, nasally, pulmonarily, mucosally, parenterally, intravascularly, intraperitoneally, subcutaneously, intramuscularly or topically. The determination of a therapeutically effective dose is well within the capability of those skilled in the art. A therapeutically effective dose is that amount sufficient to reduce symptoms, decrease nerve death, and/or increase axon length.

For any compound, the therapeutically effective dose can be estimated initially either in cell culture assays or in animal models, usually mice, rabbits, dogs, or pigs. The animal model also can be used to determine the appropriate concentration range and route of administration. Such information can then be used to determine useful doses and routes for administration in humans.

Pharmaceutical compositions which exhibit large therapeutic indices are preferred. The data obtained from cell culture assays and animal studies can be used in formulating a range of dosage for human use. The dosage contained in such compositions is preferably within a range of circulating concentrations that include the ED50 with little or no toxicity. The dosage varies within this range depending upon the dosage form employed, sensitivity of the patient, and the route of administration.

The exact dosage can be determined by the practitioner, in light of factors related to the subject that requires treatment. Dosage and administration are adjusted to provide sufficient levels of the active ingredient or to maintain the desired effect. Factors which can be taken into account include the severity of the disease state, general health of the subject, age, weight, and gender of the subject, diet, time and frequency of administration, drug combination(s), reaction sensitivities, and tolerance/response to therapy. Guidance as to particular dosages and methods of delivery is provided in the literature and generally available to practitioners in the art.

In any of the embodiments described above, any of the pharmaceutical compositions of the invention can be administered in combination with other appropriate therapeutic agents. Selection of the appropriate agents for use in combination therapy can be made by one of ordinary skill in the art, according to conventional pharmaceutical principles.

Any of the therapeutic methods described above can be applied to any subject in need of such therapy, including, for example, mammals such as dogs, cats, cows, horses, rabbits, monkeys, and most preferably, humans.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### EXAMPLES

### Example 1

PTN is upregulated in acutely denervated Schwann cells, but this upregulation is not maintained over time, and chronically denervated Schwann cells loose their ability to make PTN. Left sciatic nerve of adult rats were transected at the mid-thigh level and distal sciatic nerves were harvested at 2 and 7 days, and 1, 3 and 6 months. PTN mRNA levels were measures using real-time RT-PCR, normalized to an internal control, GAPDH and expressed as a percentage of the unoperated right side (* p<0.05 compared to the contralateral side).

### Example 2

PTN mRNA is expressed at high levels in the muscle during fetal development but expression levels decrease to minimal levels in the adult muscle. In adult muscles after denervation, PTN levels increase but return to baseline levels with re-innervation. Distal leg muscles were harvested from embryonic (day 14) and postnatal rats (6 days, 14 days, 30 days and adult) and PTN mRNA levels were measured using real-time RT-PCR.

### Example 3

PTN levels are expressed at high levels in the developing skin but expression levels are downregulated in the adulthood. There are no significant changes in the PTN mRNA levels in the skin with denervation. Plantar footpad skins were harvested from embryonic (day 14) and adult rats and PTN mRNA levels were measured using real-time RT-PCR, normalized to GAPDH and expressed as a fold difference from the adult skin levels. For denervation experiments, sciatic nerves were transected at the mid-thigh level and distal sciatic nerve innervated plantar footpad skins were harvested at 7 days and 2 months after sciatic nerve transection. (* p<0.05 compared to the adult).

### Example 4

Application of recombinant human PTN (rhPTN) as a source of neurotropism causes directional outgrowth of spinal motor axons. Spinal cord explants were prepared from P8 rats as described before (Rothstein et al., 1993; Corse et al., 1999; Ho et al., 2000). Normally under regular culture conditions, motor axons do not cross the white matter inhibitory matrix and grow out of the spinal cord. However, when PTN-soaked gelfoams were placed away from the spinal cord, they acted as a source of tropic substance allowed directional growth of motor axons (stained with SMI-32 monoclonal antibody against neurofilament). There was no directional growth with vehicle-soaked gelfoams.

### Example 5

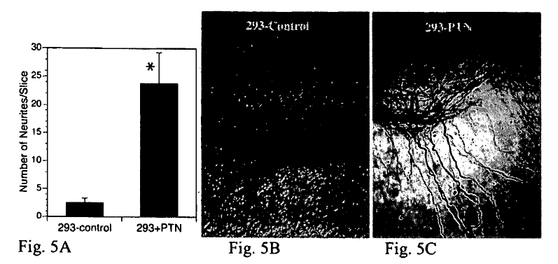

Rat PTN increases induces outgrowth of motor axons through the white matter tracts in spinal cord explants. Postnatal day 8 spinal cord explants were prepared as described above. Full-length rat PTN cDNA was cloned and HEK-293 cells were transfected with the rat PTN vector. HEK-293 cells transfected with PTN (FIG. 5C) or the vector alone (FIG. 5B) were co-cultured with the spinal cord explants for a week. At the end of the week, the explants were stained with neurofilament antibody (SMI-32) and the number of motor axons crossing the white matter and exiting from the spinal cord explants were counted. The results of the counting are shown n FIG. 5A. (* p<0.005).

### Example 6

Recombinant human PTN as well as conditioned media from HEK-293 cells transfected with PTN induced more axonal outgrowth from dorsal root ganglion (DRG) explants. Embryonic day 14 DRG were excised and cultured according to standard protocols as described previously (Keswani et al., 2003). After 48 hours of culturing the DRG explants in media containing rhPTN or in conditioned media from HEK-293 cells transfected with PTN or vector, explants were stained with anti-neurofilament antibody and the number of axons reaching at least a 100 μm away form the explant were counted (n=4 per group; GF=growth factor; * p<0.05).

### Example 7

PTN induces axonal regeneration in vivo. Based on the in vitro data using spinal cord and DRG explants, we hypothesized that PTN would also improve axonal regeneration in vitro. In order to test this hypothesis we used a rat sciatic nerve transection model. In this model, if a sciatic nerve of a rat is transected and the repair is done with silicone nerve guides but a gap of more than 10 mm is left between the proximal and distal ends, there is often no regeneration. Using this model we transplanted HEK-293 cells expressing either PTN or control plasimd into the silicone tubes and 2 months later we assessed the nerve regeneration by counting the number of axons that regenerated 15 mm distally into the silicone tube.

### Example 8

Recombinant human PTN protects motor neurons against excitotoxic cell death. Under normal conditions, glutamate transport inhibitor threo-hydroxyaspartate (THA) causes death of motor neurons in spinal cord explants due to excess glutamate (Corse et al., 1999). Postnatal day 8 spinal cord explants were cultured as above. THA with and without rhPTN were added for 4 weeks. At the end of 4 weeks, explants were stained with anti-neurofilament antibody and the number of motor neurons was counted. (* p<0.005 between control and THA; ** p<0.005 between THA and THA+PTN).

### Example 9

Recombinant human PTN protects facial motor neurons against cell death. In neonatal animals, transection of facial nerve causes motor neuron death in the facial nucleus because facial motor neurons are dependent on target derived neurotrophic factors. In an animal model the death of facial motor neurons can be prevented by other neurotrophic factors (Yan et al., 1995; Matheson et al., 1997). Using the same neonatal mouse model we performed facial axotomies in postnatal day 2 mice and transplanted gelfoams soaked with rhPTN into the stump of the facial nerve. After 1 week, we harvested the brainstems and stained with anti-neurofilament antibody to label the facial motor neurons. We counted the number of remaining facial motor neurons in each facial ganglion.

### REFERENCES

The disclosure of each reference cited is expressly incorporated herein.
Corse A M, Bilak M M, Bilak S R, Lehar M, Rothstein J D, Kuncl R W (1999) Preclinical testing of neuroprotective neurotrophic factors in a model of chronic motor neuron degeneration. Neurobiol Dis 6:335-346.
Ho T W, Bristol L A, Coccia C, Li Y, Milbrandt J, Johnson E, Jin L, Bar-Peled O, Griffin J W, Rothstein J D (2000) TGFbeta trophic factors differentially modulate motor axon outgrowth and protection from excitotoxicity. Exp Neurol 161:664-675.
Keswani S C, Polley M, Pardo C A, Griffin J W, McArthur J C, Hoke A (2003) Schwann cell chemokine receptors mediate HIV-1 gpl20 toxicity to sensory neurons. Ann Neurol 54:287-296.
Matheson C R, Wang J, Collins F D, Yan Q (1997) Long-term survival effects of GDNF on neonatal rat facial motoneurons after axotomy. Neuroreport 8:1739-1742.
Rothstein J D, Jin L, Dykes-Hoberg M, Kuncl R W (1993) Chronic inhibition of glutamate uptake produces a model of slow neurotoxicity. Proc Natl Acad Sci USA 90:6591-6595.
Yan Q, Matheson C, Lopez O T (1995) In vivo neurotrophic effects of GDNF on neonatal and adult facial motor neurons. Nature 373:341-344.

1. A method of treating a peripheral neuropathy, peripheral neurodegenerative disease, or peripheral nerve trauma, comprising:
administering an effective amount of a nucleic acid encoding pleiotrophin to a patient with a peripheral neuropathy, peripheral neurodegenerative disease, or peripheral nerve trauma, whereby axonal regeneration is stimulated or neuronal death is inhibited.

2. The method of claim 1 wherein the nucleic acid is a viral nucleic acid vector.

3. The method of claim 1 wherein the nucleic acid is a plasmid vector.

4. The method of claim 1 wherein the peripheral neuropathy, peripheral neurodegenerative disease, or peripheral nerve trauma is metabolic neuropathy.

5. The method of claim 3 wherein the metabolic neuropathy is diabetic neuropathy.

6. The method of claim 3 wherein the metabolic neuropathy is uremic neuropathy.

7. The method of claim 1 wherein the peripheral neuropathy, peripheral neurodegenerative disease, or peripheral nerve trauma is toxic neuropathy.

8. The method of claim 7 wherein the toxic neuropathy is chemotherapy-induced neuropathy.

9. The method of claim 7 wherein the toxic neuropathy is anti-retroviral drug-induced neuropathy.

10. The method of claim 1 wherein the peripheral neuropathy, peripheral neurodegenerative disease, or peripheral nerve trauma is a motor neuron disease.

11. The method of claim 10 wherein the motor neuron disease is amyotrophic lateral sclerosis.

12. The method of claim 10 wherein the motor neuron disease is spinobulbar muscular atrophy.

13. The method of claim 10 wherein the motor neuron disease is spinal muscular atrophy.

14. The method of claim 1 wherein the peripheral neuropathy, peripheral neurodegenerative disease, or peripheral nerve trauma is Charcoat-Marie-Tooth disease.

15. The method of claim 1 wherein the peripheral neuropathy, peripheral neurodegenerative disease, or peripheral nerve trauma is peripheral nerve trauma, and the peripheral nerve trauma is caused by injury.

16. The method of claim 15 wherein the injury is open.

17. The method of claim 15 wherein the injury is closed.

18. The method of claim 1 wherein the nucleic acid is adeno-associated virus.

19. The method of claim 1 wherein the step of administering is by injection into muscle of the patient.

20. The method of claim 18 wherein the step of administering is by injection into muscle of the patient.
